(19) United States
(12) Patent Application Publication
(10) Pub. No.: US 2020/0154989 A1
(43) Pub. Date: May 21, 2020

(54) ENDOSCOPE SYSTEM AND LIGHT SOURCE DEVICE FOR ENDOSCOPE

(71) Applicant: OLYMPUS CORPORATION, Tokyo (JP)

(72) Inventors: Satoshi OHARA, Hachioji-shi (JP); Takeshi ITO, Hino-shi (JP)

(73) Assignee: OLYMPUS CORPORATION, Tokyo (JP)

(21) Appl. No.: 16/749,446

(22) Filed: Jan. 22, 2020

Related U.S. Application Data
(63) Continuation of application No. PCT/JP2017/027042, filed on Jul. 26, 2017.

(51) Int. Cl. A61B 1/06; A61B 1/00

(52) U.S. Cl. CPC A61B 1/0653; A61B 1/00167; A61B 1/00013; A61B 1/00096

(57) ABSTRACT

An endoscope system includes: an excitation light source to emit excitation light; non-excitation light sources to emit non-excitation light; an optical combiner configured to integrate optical paths of the excitation light and non-excitation light into a common optical path; and a wavelength conversion unit including a wavelength conversion member disposed on the common optical path. The wavelength conversion member transmits the non-excitation light, absorbs part of the excitation light to generate wavelength-converted light, and emits illumination light including the non-excitation light and wavelength-converted light. Spectra of the non-excitation light each have a peak wavelength in a wavelength band out of a wavelength band of a spectrum of the wavelength-converted light.

### CROSS-REFERENCE TO RELATED APPLICATIONS

This application is a Continuation Application of PCT Application No. PCT/JP2017/027042, filed Jul. 26, 2017, the entire contents of which are incorporated herein by reference.

### BACKGROUND OF THE INVENTION

### 1. Field of the Invention

The present invention relates to an endoscope system and a light source device for endoscopes.

### 2. Description of the Related Art

Japanese Patent No. 5019289 discloses an example of an illumination device used in an endoscope system. The illumination device includes a plurality of light sources configured to emit respective rays of excitation light and non-excitation light having a plurality of wavelengths, a plurality of single fibers configured to guide the respective rays of excitation light and non-excitation light, a plurality of fluorescent substance units irradiated with respective rays of excitation light emitted from respective single fibers, and a fiber bundle configured to guide fluorescent light generated from the fluorescent substance units and the non-excitation light not radiated to the fluorescent substance units.

The fluorescent substance units are disposed in parallel with each other. The fiber bundle has an end on the side of the fluorescent substance units divided into a plurality of ends and is mounted inside the endoscope system so that the divided ends are optically coupled to the respective fluorescent substance units.

### BRIEF SUMMARY OF THE INVENTION

An endoscope system includes: at least one excitation light source configured to emit excitation light; at least two non-excitation light sources configured to respectively emit at least two rays of non-excitation light; an optical combiner configured to integrate an optical path of the excitation light and optical paths of the non-excitation light into a common optical path; and a wavelength conversion unit including a wavelength conversion member disposed on the common optical path. The wavelength conversion member is configured to: transmit the non-excitation light; absorb components of part of the excitation light to generate wavelength-converted light having a wavelength different from a wavelength of the excitation light; and emit illumination light including the transmitted non-excitation light and the generated wavelength-converted light. Spectra of the at least two rays of non-excitation light each have a peak wavelength in a wavelength band out of a wavelength band of a spectrum of the wavelength-converted light.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 shows an endoscope system according to an embodiment.

FIG. 2 shows a block diagram of the endoscope system shown in FIG. 1.

FIG. 3 schematically shows a configuration example of an illumination device of the endoscope system shown in FIG. 2.

FIG. 4 shows spectra of laser light respectively emitted from laser light sources shown in FIG. 3 and a spectrum of fluorescent light generated from a fluorescent substance shown in FIG. 3.

FIG. 5 schematically shows another configuration example of the illumination device of the endoscope system shown in FIG. 2.

FIG. 6 schematically shows another configuration example of the illumination device of the endoscope system shown in FIG. 2.

### DETAILED DESCRIPTION OF THE INVENTION

[Endoscope System]

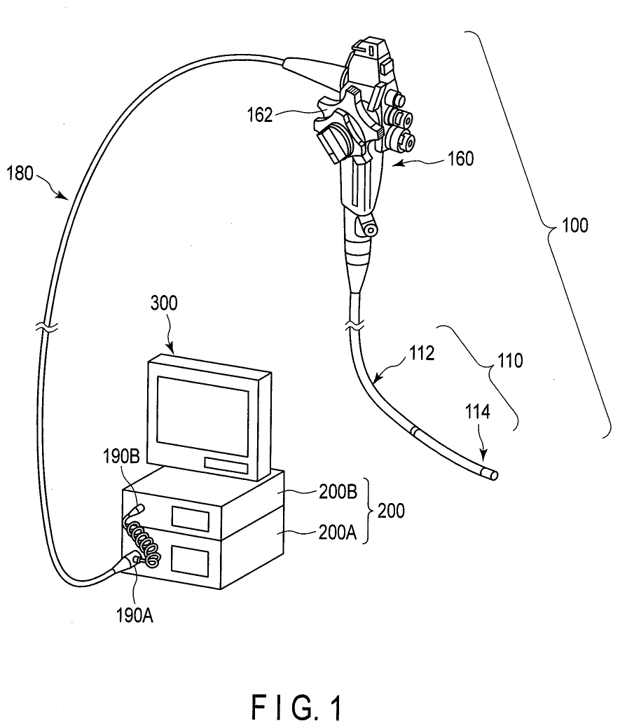

FIG. 1 shows an endoscope system according to an embodiment. The endoscope system includes a scope 100 configured to illuminate an observation object and acquire an image of the observation object, a control box 200 configured to control the endoscope system, and a monitor 300 configured to display the acquired image of the observation object.

<Scope 100>

The scope 100 includes a hollow elongate insertion section 110 that is inserted into a tube hole of the observation object, a control section 160 that is coupled to a proximal end section of the insertion section 110, and a connection cable 180 that extends from the control section 160.

The insertion section 110 includes a bendable section 112 configured to be flexible and a distal end section 114 configured to be rigid. Thereby, the bendable section 112 is configured to be able to passively bend. For example, the bendable section 112 bends following a shape inside the tube hole when inserted into the tube hole of the observation object.

The control section 160 is provided with a control handle 162 configured to bend the insertion section 110 in a vertical direction and a horizontal direction. The insertion section 110 is bent in the vertical direction and horizontal direction according to operation of the control handle 162 by an operator. That is, the insertion section 110 is configured to be able to actively bend.

<Control Box 200>

The control box 200 includes a light source box 200A configured to supply illumination light, and a circuit box 200B incorporating various electronic circuits necessary for control of the endoscope system.

The scope 100 is connected to the control box 200 through the connection cable 180. At an end of the connection cable 180, a scope connector 190A for optical connection and a scope connector 190B for electrical connection are provided. The connection cable 180 is optically connected to the light source box 200A by the scope connector 190A and is electrically connected to the circuit box 200B by the scope connector 190B.

<Monitor 300>

The monitor 300 may comprise, but not limited to, a liquid crystal display, for example.

[Illumination Device and Imaging Device]

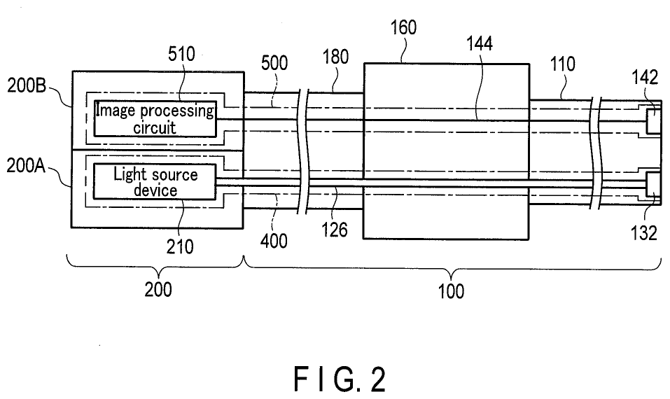

FIG. 2 shows a block diagram of the endoscope system shown in FIG. 1. The endoscope system includes an illumination device 400 configured to illuminate the observation object and an imaging device 500 configured to take images of the observation object.

<Illumination Device 400>

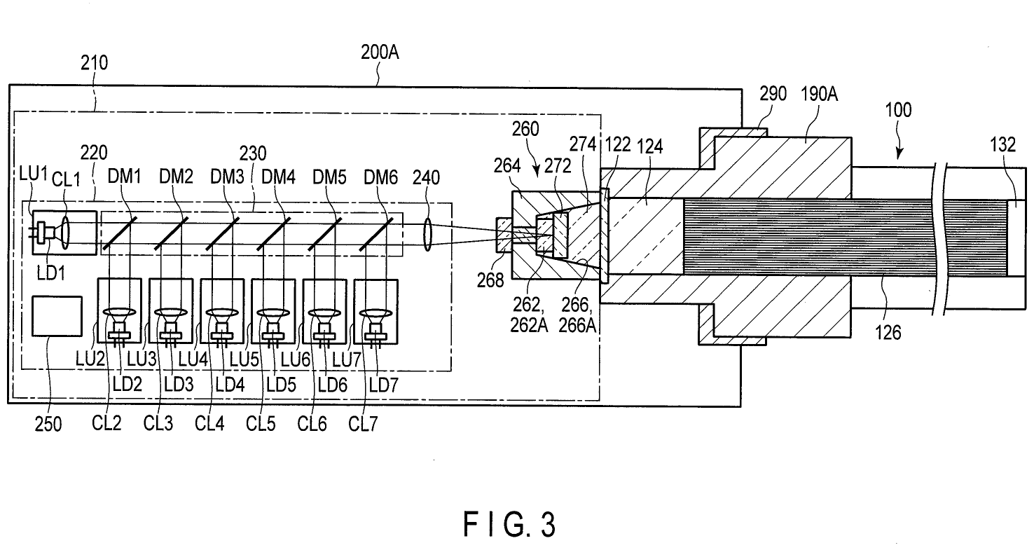

FIG. 3 schematically shows a configuration example of the illumination device 400 of the endoscope system shown in FIG. 2. The illumination device 400 according to this configuration example includes a light source device 210 configured to emit illumination light for illuminating the observation object, a light guide lens 124 that the illumination light emitted from the light source device 210 enters, a cover glass 122 provided so as to protect an end surface of the light guide lens 124, a light guide 126 configured to guide the illumination light emitted from the light source device 210, and an illumination light emission unit 132 configured to emit the illumination light guided by the light guide 126 to the outside of the scope 100.

The light source device 210 is disposed inside the light source box 200A. The cover glass 122, the light guide lens 124, the light guide 126, and the illumination light emission unit 132 are disposed inside the scope 100. That is, in other words, the light source device 210 constitutes the illumination device 400 in cooperation with the scope 100.

The cover glass 122 and the light guide lens 124 are held by a scope connector 190A that is attachable to and detachable from a connector receiver 290 provided in the light source box 200A.

The light guide lens 124 is a cylindrical lens having a core/cladding structure and has a function of causing intensity distribution of the illumination light entered from the light source device 210 uniform.

The light guide 126 extends to the inside of the scope 100 with an end held by the scope connector 190A.

In more detail, the light guide 126 extends from the scope connector 190A to the distal end section 114 through the inside of the connection cable 180, the control section 160, and the insertion section 110. The light guide 126 comprises, for example, a bundle fiber in which a large number of extremely thin optical fibers are bundled. Alternatively, the light guide 126 may comprise a single optical fiber.

The illumination light emission unit 132 is optically connected to the light guide 126 and is disposed at the distal end section 114 of the insertion section 110.

The illumination light emitted from the light source device 210 enters the light guide 126 through the cover glass 122 and the light guide lens 124. Then, the illumination light is guided by the light guide 126 to enter the illumination light emission unit 132. Subsequently, the illumination light is emitted to the outside of the scope 100 by the illumination light emission unit 132. The illumination light emitted to the outside of the scope 100 is radiated, for example, to the observation object. The illumination light radiated to the observation object is, for example, reflected or scattered by the observation object.

<Imaging Device>

As shown in FIG. 2, for example, the imaging device 500 includes an imager 142 configured to receive light (reflected light and scattered light) from the observation object to acquire an optical image of the observation object, and an image processing circuit 510 configured to process an image signal of the optical image of the observation object acquired by the imager 142. The imager 142 is installed at the distal end section 114 of the insertion section 110. The image processing circuit 510 is disposed inside the circuit box 200B. The imager 142 is electrically connected to the image processing circuit through an electric signal line 144.

The image signal of the optical image of the observation object acquired by the imager 142 is supplied to the image processing circuit 510. The image processing circuit 510 applies necessary image processing to the supplied image signal and supplies the image-processed image signal to the monitor 300. The monitor 300 displays an image according to the supplied image signal.

[Light Source Device 210]

The light source device 210 includes a light source unit 220 configured to be able to emit excitation light and non-excitation light and a wavelength conversion unit (wavelength converter) 260 configured to generate wavelength-converted light from the excitation light.

<Light Source Unit 220>

(Laser Units LU1-LU7)

The light source unit 220 is configured to be able to emit light having seven wavelengths as an example in the embodiment. Therefore, the light source unit 220 includes seven laser units LU1-LU7 configured to emit laser light. Here, wavelengths of the laser light emitted by the laser units LU1-LU7 are different from each other.

The laser units LU1-LU7 include laser light sources LD1-LD7 and collimating lenses CL1-CL7, respectively. The laser light sources LD1-LD7 may comprise, but not limited to, laser diodes, for example. The laser light sources LD1-LD7 are, for example, as follows.

The laser light source LD1 is a violet laser light source configured to emit violet laser light. A spectrum of the violet laser light emitted from the laser light source LD1 has, for example, a wavelength band of 390-445 nm and a peak wavelength at approximately 415 nm. Alternatively, the spectrum of the violet laser light emitted from the laser light source LD1 has a wavelength band of 390-470 nm and a peak wavelength at 430 nm.

The laser light source LD2 is a blue laser light source configured to emit blue laser light. A spectrum of the blue laser light emitted from the laser light source LD2 has, for example, a wavelength band of 435-455 nm and a peak wavelength at 445 nm.

The laser light source LD3 is a green laser light source configured to emit green laser light. A spectrum of the green laser light emitted from the laser light source LD3 has, for example, a wavelength band of 530-550 nm and a peak wavelength at 540 nm. Alternatively, the spectrum of the green laser light emitted from the laser light source LD3 has a wavelength band of 540-560 nm and a peak wavelength at 550 nm.

The laser light source LD4 is an orange laser light source configured to emit orange laser light. A spectrum of the orange laser light emitted from the laser light source LD4 has, for example, a wavelength band of 600-630 nm and a peak wavelength at 615 nm.

The laser light source LD5 is a red laser light source configured to emit red laser light. A spectrum of the red laser light emitted from the laser light source LD5 has, for example, a wavelength band of 680-700 nm and a peak wavelength at 690 nm.

The laser light source LD6 is an infrared laser light source configured to emit infrared laser light. A spectrum of the infrared laser light emitted from the laser light source LD6 has, for example, a wavelength band of 790-820 nm and a peak wavelength at 805 nm.

The laser light source LD7 is an infrared laser light source configured to emit infrared laser light. A spectrum of the infrared laser light emitted from the laser light source LD7 has, for example, a wavelength band of 905-970 nm and a peak wavelength at approximately 935 nm.

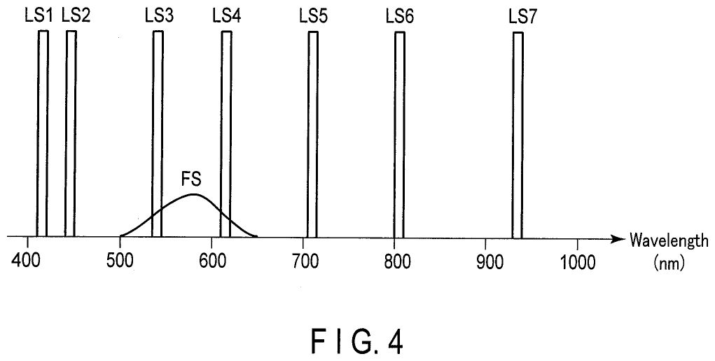

FIG. 4 shows spectra LS1-LS7 of the laser light emitted from the laser light sources LD1-LD7, respectively. The spectra LS1-LS7 shown in FIG. 4 represent only relative magnitude relationships among the peak wavelengths and width of the wavelength bands is not accurately reflected in favor of easy viewing. FIG. 4 also shows a spectrum FS of fluorescent light emitted from a fluorescent substance 262A described later. The spectrum FS of the fluorescent light has a wavelength band of 500-650 nm and has a peak wavelength at 580 nm.

As described later, the wavelength conversion unit 260 includes a wavelength conversion member 262 configured to absorb excitation light to generate wavelength-converted light. For example, the wavelength conversion member 262 comprises the fluorescent substance 262A configured to absorb excitation light to generate fluorescent light as the wavelength-converted light. For example, the fluorescent substance 262A comprises a yellow fluorescent substance using YAG. The YAG has an absorption wavelength range in a wavelength region of blue light.

The wavelength band of the laser light emitted from the laser light source LD2 matches the absorption wavelength band of the wavelength conversion member 262, for example, the fluorescent substance 262A, and particularly the yellow fluorescent substance. On the other hand, the wavelength bands of the laser light emitted from the laser light sources LD1 and LD3-LD7 do not match the absorption wavelength band of the wavelength conversion member 262, for example, the fluorescent substance 262A, and particularly the yellow fluorescent substance. That is, the laser light source LD2 is an excitation light source configured to emit excitation light. On the other hand, the laser light sources LD1 and LD3-LD7 are non-excitation light sources configured to emit non-excitation light.

The non-excitation light emitted from each of the laser light sources LD1 and LD3-LD7 may preferably be narrowband light. The use of the narrowband light will lead the performance of special light observation to improve.

In other words, the laser light sources LD1 and LD3-LD7 may be narrowband light sources. Furthermore, the laser light source LD2 may also be a narrowband light source. Although the narrowband light sources comprise the laser light sources LD1-LD7 in the embodiment, they are not limited to this, and may comprise LEDs or the like.

LEDs emit light having a considerably narrow wavelength band although the band is not as narrow as that of laser light sources. Therefore, the light generated by LEDs can be regarded as narrowband light. Narrowband light sources are not limited to laser light sources and any kind of light sources may be applied as long as they emit light that can be regarded as narrowband light like LEDs.

The spectra LS1 and LS3-LS7 of the laser light emitted from the respective laser light sources LD1 and LD3-LD7, which are non-excitation light sources, have the following relationships with the spectrum FS of the fluorescent light and the spectrum LS2 of the laser light emitted from the laser light source LD2, which is an excitation light source.

AS shown in FIG. 4, the spectra LS1 and LS3 each have a peak wavelength shorter than a peak wavelength of the spectrum FS of the fluorescent light. On the other hand, the spectra LS4-LS7 each have a peak wavelength longer than the peak wavelength of the spectrum FS of the fluorescent light.

The spectra LS1, LS5, LS6, and LS7 each have a peak wavelength out of the spectrum FS of the fluorescent light. Furthermore, the spectra LS1, LS5, LS6, and LS7 are out of the spectrum FS of the fluorescent light. In other words, the spectra LS1, LS5, LS6 and LS7 do not overlap with the spectrum FS of the fluorescent light. On the other hand, the spectra LS3 and LS4 each have a peak wavelength situated in a wavelength range of the spectrum FS of the fluorescent light. Furthermore, the spectra LS3 and LS4 entirely overlap with the spectrum FS of the fluorescent light. In other words, the wavelength bands of the spectra LS3 and LS4 are situated in the wavelength band of the spectrum FS of the fluorescent light.

The spectrum LS1 has the peak wavelength shorter than the peak wavelength of the spectrum LS2 of the laser light emitted from the laser light source LD2, an excitation light source. On the other hand, the spectra LS3-LS7 each have the peak wavelength longer than the peak wavelength of the spectrum LS2.

At least, the light source unit 220 includes at least one excitation light source and at least one non-excitation light source. Specifically, the light source unit 220 includes one excitation light source, that is, the laser light source LD2, and six non-excitation light sources, that is, the laser light sources LD1 and LD3-LD7. Although the light source unit 220 includes only one excitation light source in the embodiment, it may include a plurality of excitation light sources. In addition, although the light source unit 220 includes a plurality of non-excitation light sources, it may include only one non-excitation light source.

The light source unit 220 may include some of the six laser light sources LD1 and LD3-LD7, non-excitation light sources, or may further include another non-excitation light source, for example, a laser light source, in addition to the six laser light sources LD1 and LD3-LD7, non-excitation light sources.

For example, the light source unit 220 may include a plurality of non-excitation light sources as described below.

The light source unit 220 includes at least two non-excitation light sources (for example, the laser light sources LD1, LD5, LD6, and LD7 configured to emit the non-excitation light of the spectra LS1, LS5, LS6, and LS7) configured to emit non-excitation light of spectra each having a peak wavelength out of the wavelength band of the spectrum FS of the fluorescent light.

The light source unit 220 includes at least a non-excitation light source (for example, the laser light source LD3 configured to emit the non-excitation light of the spectrum LS3) configured to emit non-excitation light of a spectrum having a peak wavelength that is shorter than the peak wavelength of the spectrum FS of the fluorescent light and situated in the wavelength band of the spectrum FS of the fluorescent light, and a non-excitation light source (for example, the laser light source LD4 configured to emit the non-excitation light of the spectrum LS4) configured to emit non-excitation light of a spectrum having a peak wavelength that is longer than the peak wavelength of the spectrum FS of the fluorescent light and situated in the wavelength band of the spectrum FS of the fluorescent light.

The light source unit 220 includes at least a non-excitation light source (for example, the laser light source LD1 configured to emit the non-excitation light of the spectrum LS1) configured to emit non-excitation light of a spectrum having a peak wavelength that is shorter than the peak wavelength of the spectrum LS2 of excitation light and shorter than the longest wavelength of the spectrum FS of the fluorescent light, and a non-excitation light source (for example, the laser light sources LD3 and LD4 configured to emit the non-excitation light of the spectra L3 and LS4) configured to emit non-excitation light of a spectrum having a peak wavelength that is longer than the peak wavelength of the spectrum LS2 of excitation light and shorter than the longest wavelength of the spectrum FS of the fluorescent light.

As described before, the laser units LU1-LU7 include the collimating lenses CL1-CL7 in addition to the laser light sources LD1-LD7, respectively. The collimating lenses CL1-CL7 are configured to change divergent beams of the laser light emitted from the laser light sources LD1-LD7 into parallel beams, respectively. In the laser light sources LD1-LD7, the size of light emitting points and light distributions of beams of laser light are different from each other. Consequently, if the divergent beams of the laser light are changed into the parallel beams under the same conditions for the collimating lenses CL1-CL7 and laser light sources LD1-LD7, diameters of the parallel beams are different from each other. The collimating lenses CL1-CL7 are preferably designed tailored to characteristics of the respective laser light sources LD1-LD7 so that the diameters of all the parallel beams are the same. In addition, the light distribution of light refers to how the beam of light spreads.

(Optical Combiner 230)

The light source unit 220 also includes an optical combiner 230 configured to integrate seven optical paths of the laser light emitted from the laser units LU1-LU7 into one common optical path.

The optical combiner 230 includes six dichroic mirrors DM1-DM6. The dichroic mirrors DM1-DM6 are configured as follows.

The dichroic mirror DM1 is configured to transmit the laser light emitted from the laser light source LD1 but reflect the laser light emitted from the laser light source LD2. The dichroic mirror DM1 is disposed so that an optical path of the transmitted laser light and an optical path of the reflected laser light are integrated into one optical path with having a common axis.

The dichroic mirror DM2 is configured to transmit the laser light emitted from the laser light sources LD1 and LD2 but reflect the laser light emitted from the laser light source LD3. The dichroic mirror DM2 is disposed so that an optical path of the transmitted laser light and an optical path of the reflected laser light are integrated into one optical path with having a common axis.

The dichroic mirror DM3 is configured to transmit the laser light emitted from the laser light sources LD1-LD3 but reflect the laser light emitted from the laser light source LD4. The dichroic mirror DM3 is disposed so that an optical path of the transmitted laser light and an optical path of the reflected laser light are integrated into one optical path with having a common axis.

The dichroic mirror DM4 is configured to transmit the laser light emitted from the laser light sources LD1-LD4 but reflect the laser light emitted from the laser light source LD5. The dichroic mirror DM4 is disposed so that an optical path of the transmitted laser light and an optical path of the reflected laser light are integrated into one optical path with having a common axis.

The dichroic mirror DM5 is configured to transmit the laser light emitted from the laser light sources LD1-LD5 but reflect the laser light emitted from the laser light source LD6. The dichroic mirror DM5 is disposed so that an optical path of the transmitted laser light and an optical path of the reflected laser light are integrated into one optical path with having a common axis.

The dichroic mirror DM6 is configured to transmit the laser light emitted from the laser light sources LD1-LD6 but reflect the laser light emitted from the laser light source LD7. The dichroic mirror DM6 is disposed so that an optical path of the transmitted laser light and an optical path of the reflected laser light are integrated into one optical path with having a common axis.

Consequently, the optical combiner 230 is configured to integrate seven optical paths of the laser light emitted from the laser light sources LD1-LD7 into one common optical path. In other words, the optical combiner 230 is configured to integrate one optical path of the excitation light emitted from the laser light source LD2 and six optical paths of the non-excitation light emitted from the laser light sources LD1 and LD3-LD7 into one common optical path. Furthermore, the optical combiner 230 integrates one optical path of the excitation light emitted from the laser light source LD2 and six optical paths of the non-excitation light emitted from the laser light sources LD1 and LD3-LD7 into one common optical path so that their optical axes are substantially coincident and their diameters are substantially coincident.

(Condenser Lens 240)

The light source unit 220 also includes a condenser lens 240 disposed on an optical path of laser light emitted from the optical combiner 230, that is, on the common optical path. The condenser lens 240 is configured to converge the laser light emitted from the optical combiner 230. A parallel beam of the laser light emitted from the optical combiner 230 is changed into a converging beam by the condenser lens 240, and the converging beam is emitted from the light source unit 220 and enters the wavelength conversion unit 260 while its diameter is reducing.

(Light Source Controller 250).

The light source unit 220 also includes a light source controller 250 configured to control drive of the laser light sources LD1-LD7. The light source controller 250 is configured to adjust quantities of light of the laser light sources LD1-LD7 by acquiring and analyzing an image signal from the image processing circuit so that, for example, an image of the observation object is displayed on the monitor 300 at an appropriate luminance.

[Wavelength Conversion Unit 260]

The light source device 210 includes the wavelength conversion unit 260 in addition to the light source unit 220 as described before. The light source device 210 is disposed inside the light source box 200A as described before. Consequently, the wavelength conversion unit 260 is disposed inside the light source box 200A. In other words, the wavelength conversion unit 260 is disposed outside the scope 100.

The wavelength conversion unit 260 includes the wavelength conversion member 262 configured to absorb components of part of excitation light to generate wavelength-converted light having a wavelength different from a wavelength of the excitation light, a diffuser 272 configured to diffuse radiated light to expand its light distribution, a wavelength filter 268 configured to transmit only light having a specific wavelength, and a holder 264 configured to hold the wavelength conversion member 262, the diffuser 272, and the wavelength filter 268 at predetermined positions.

The wavelength conversion member 262, the diffuser 272, and the wavelength filter 268 are disposed on the optical path of the laser light emitted from the light source unit 220, that is, on the common optical path.

<Wavelength Conversion Member 262>

The wavelength conversion member 262 is configured to transmit non-excitation light, absorb components of part of excitation light to generate wavelength-converted light having a wavelength different from a wavelength of the excitation light, and emit illumination light including the transmitted non-excitation light and/or the generated wavelength-converted light. The illumination light emitted from the wavelength conversion member 262 may include the non-excitation light in addition to the wavelength-converted light. For example, the wavelength conversion member 262 is configured to emit illumination light including a plurality of rays of non-excitation light, for example, at least two or three rays of non-excitation light. Also, the wavelength conversion member 262 is configured so that transmittance is higher in a wavelength range of non-excitation light than in a wavelength range of excitation light.

The wavelength conversion member 262 comprises the fluorescent substance 262A, for example. The fluorescent substance 262A is configured to absorb components of part of radiated excitation light to generate fluorescent light having a wavelength longer than a wavelength of the excitation light. The fluorescent light generated by the fluorescent substance 262A includes a forward fluorescent component and a rearward fluorescent component. The forward fluorescent component is a fluorescent component that proceeds toward the scope 100 whereas the rearward fluorescent component is a fluorescent component that proceeds toward the light source unit 220. In addition, the fluorescent light has a wider spectrum than a spectrum of excitation light.

<Phosphor 262A>

The fluorescent substance 262A is, for example, a yellow fluorescent substance configured to absorb some component of 445-nm blue laser light to isotropically generate yellow fluorescent light. The yellow fluorescent light generated from the fluorescent substance 262A has a broad spectrum FS extending from green to orange as shown in FIG. 4. The spectrum FS of the yellow fluorescent light has, for example, a wavelength band of 500-650 nm and a peak wavelength at 580 nm as described before. Specifically, the yellow fluorescent substance is a fluorescent substance indicated by a composition of YAG (Y3AL5O12: Ce). In the embodiment, the fluorescent substance 262A includes a fluorescent substance made of a poly-crystallized YAG ceramic, has a property of hardly diffusing transmitted excitation light, and has a high thermal conductivity of approximately 10 W/mK. For the fluorescent substance 262A, for example, even if a YAG single crystal is used, a powdery YAG fluorescent substance is dispersed on a sealing material such as glass or silicone resin and the sealing material is solidified, thereby forming a thing, and that thing may be used.

The fluorescent substance 262A has an efficiency (internal quantum efficiency) of converting a quantity of light of absorbed excitation light into fluorescent light and the internal quantum efficiency has a predetermined value. Specifically, the fluorescent substance 262A has an internal quantum efficiency of approximately 80%. Therefore, when the fluorescent substance 262A converts the wavelength, the quantity of light corresponding to approximately 80% of a quantity of light of the absorbed excitation light is wavelength-converted, whereas the quantity of light corresponding to approximately 20% is lost and converted into heat. In this way, the fluorescent substance 262A has a property of simultaneously generating heat corresponding to conversion loss when converting the wavelength. Furthermore, the shape of the fluorescent substance 262A is, for example, cylindrical.

<Diffuser 272>

The diffuser 272 has, for example, a function of diffusing excitation light and non-excitation light that have entered and expanding light distributions of the excitation light and non-excitation. Diffused light generated by the diffuser 272 includes a forward-scattered light component and a backscattered light component. The forward-scattered light component is a scattered light component that proceeds toward the scope 100 whereas the backscattered light component is a scattered light component that proceeds toward the light source unit 220. The diffuser 272 is preferably disposed at a later stage than the wavelength conversion member 262. This is because the backscattered light component generated by the diffuser 272 enters the wavelength conversion member 262 again and is absorbed, thereby increasing the wavelength-converted light generated by the wavelength conversion member 262.

The diffuser 272 is formed, for example, by mixing and curing diffusion particles having a refractive index different from that of a transparent resin with the transparent resin having a high transmittance at wavelengths of excitation light, non-excitation light, and fluorescent light. Specifically, a silicone resin, an epoxy resin, or the like is selected as the transparent resin. The diffusion particles have a refractive index far from that of the transparent resin, and have a property of causing a diffusion phenomenon when light enters the diffusion particles. Therefore, the diffusion particles are made of material such as alumina or titanium oxide, and the particle size is preferably several μm or so. Although the shape of the diffuser 272 is cylindrical in the embodiment, it may be a dome shape. The dome-shaped diffuser 272 is formed, for example, by applying material before curing to the fluorescent substance 262A and then curing it. By controlling the quality of material, diameter, and mixed concentration of the diffusion particles and the thickness of the diffuser 272, an appropriate degree of diffusion can be adjusted.

<Wavelength Filter 268>

The wavelength filter 268 is designed so as to transmit excitation light and non-excitation light but reflect light having other frequencies, for example, wavelength-converted light (for example, fluorescent light). The wavelength filter 268 specifically includes a dielectric multilayer film, and the transmission wavelength and reflection wavelength can be controlled by designing the quality of material and thickness of thin films to be laminated.

In the embodiment, the wavelength filter 268 is disposed at a portion where light (that is, excitation light and non-excitation light) enters the wavelength conversion unit 260 from the light source unit 220. In addition, the wavelength filter 268 does not obstruct the excitation light and non-excitation light from entering but reflects part of the fluorescent light generated inside the wavelength conversion unit 260. Thereby, the wavelength filter 268 contributes to improving the conversion efficiency of the wavelength conversion unit 260.

<Holder 264>

The holder 264 includes an entrance hole on the side facing the light source unit 220 and an exit hole on the side facing the light guide 126, and has a substantially cylindrical shape inside which the two holes are communicated with each other. The holder 264 holds the fluorescent substance 262A and the diffuser 272 at predetermined positions inside the cylinder, and other spaces are filled with a transparent resin 274. In the embodiment, the wavelength filter 268 is disposed outside an entrance hole portion of the holder 264. Of course, there is no problem either with a design in which the wavelength filter 268 is disposed inside the holder 264.

An inner wall of the cylindrical holder 264 is tapered-shaped and is designed to cause the excitation light, fluorescent light, and non-excitation light efficiently to enter the light guide 126. Furthermore, a cylindrical inner wall surface is provided with a reflection coating 266 made of material, for example, silver or aluminum, in order to efficiently collect the excitation light, fluorescent light, and non-excitation light in a direction of the exit hole.

The reflection coating 266 constitutes a reflector 266A configured to control the light distributions of the excitation light, non-excitation light, and wavelength-converted light in cooperation with the holder 264. The reflector 266A has a function of causing the light distributions of the excitation light, non-excitation light, and wavelength-converted light (for example, fluorescent light) emitted from the wavelength conversion unit 260 to be equal to or less than a predetermined spread angle. For example, the reflector 266A has a function of reflecting the backscattered light component to combine it with the forward-scattered light component, so as to cause the light distribution of the diffused light emitted from the wavelength conversion unit 260 to be equal to or less than the predetermined spread angle. The reflector 266A also has a function of reflecting the rearward fluorescent component to combine it with the forward fluorescent component, so as to cause the light distribution of the fluorescent light emitted from the wavelength conversion unit 260 to be equal to or less than the predetermined spread angle. Furthermore, the reflector 266A has a function of substantially matching the light distributions of the excitation light, non-excitation light, and wavelength-converted light (for example, fluorescent light) emitted from the wavelength conversion unit 260. Consequently, the light distribution of the forward-scattered light component is matched with the light distribution of the forward fluorescent component. Properties of the reflector 266A are controlled by the shape of the inner wall surface of the holder 264 provided with the reflection coating 266.

The reflector 266A has the function of causing the light distributions of the excitation light, non-excitation light, and wavelength-converted light, that is, fluorescent light emitted from the wavelength conversion unit to be equal to or less than the predetermined spread angle. For example, the reflector 266A reflects the backscattered light component to combine it with the forward-scattered light component, so as to cause the light distribution of the diffused light emitted from the wavelength conversion unit 260 to be equal to or less than the predetermined spread angle. The reflector 266A also reflects the rearward fluorescent component to combine it with the forward fluorescent component, so as to cause the light distribution of the fluorescent light emitted from the wavelength conversion unit 260 to be equal to or less than the predetermined spread angle. Furthermore, the reflector 266A has a function of substantially matching the light distributions of the excitation light, non-excitation light, and wavelength-converted light, that is, fluorescent light emitted from the wavelength conversion unit 260.

The holder 264 has, on part of the inner wall, a stop surface that is a flat surface for abutting and positioning the fluorescent substance 262A against it.

Since the holder 264 is a member holding the fluorescent substance 262A, the holder 264 is made from material excellent in heat conduction, such as copper, aluminum, brass, and aluminum nitride, which has a high effect of dispersing heat generated by the fluorescent substance 262A.

<Optical Path, Wavelength Conversion, Light Distribution Adjustment, and Aperture Number inside Wavelength Conversion Unit 260>

Describing the optical path in order, first, a beam of the excitation light and/or non-excitation light emitted from the light source unit 220 enter the wavelength conversion unit 260 while reducing their diameters. The excitation light and/or non-excitation light pass through the wavelength filter 268, and proceed from the entrance hole of the holder 264 to the inside of the holder 264. Reducing the beam diameter of the excitation light and/or non-excitation light as much as possible here allows the diameter of the entrance hole of the holder 264 to be reduced, so that a quantity of fluorescent light leaking rearward described later can be reduced.

The excitation light and/or non-excitation light are radiated to the fluorescent substance 262A inside the holder 264. Components of part of the excitation light are absorbed by the fluorescent substance 262A, and approximately 80% of absorbed energy is converted into fluorescent light. The fluorescent light is emitted isotropically in all directions. That is, the fluorescent light includes a forward fluorescent component and a rearward fluorescent component with substantially equal quantities of light. The remaining approximately 20% is converted into heat and does not contribute to illumination.

On the other hand, components of part of the excitation light radiated to the fluorescent substance 262A pass through the fluorescent substance 262A as is, and enters the diffuser 272. Furthermore, the non-excitation light similarly enters the fluorescent substance 262A, passes through the fluorescent substance 262A without being absorbed, and enters the diffuser 272. Since the fluorescent substance 262A made of YAG ceramic used in the embodiment does not have a diffusion function, the light distribution of the excitation light and/or non-excitation light that have passed through still has a small spread angle.

Microscopically, the excitation light and/or non-excitation light that have entered the diffuser 272 are mainly scattered forward and rearward by a diffusion phenomenon caused by the diffusion particles mixed inside the diffuser 272. This microscopic phenomenon occurs in all the diffusion particles, but macroscopically, it may be considered that the diffuser 272 has a function of scattering light forward and rearward in the same manner as the diffusion particles. That is, the excitation light and non-excitation light that have entered the diffuser 272 are converted into diffused light. The diffused light includes a forward-scattered light component and a backscattered light component. The light distribution of the diffused light of the forward-scattered light component is, for example, substantially Lambertian light distribution, which is a sufficiently wide light distribution.

Components of part of the excitation light and fluorescent light and/or non-excitation light are reflected by the reflection coating 266 provided on the inner wall surface of the holder 264. At this time, since the inner wall surface of the holder 264 has a tapered shape whose diameter increases as it approaches the exit hole from the entrance hole, the reflected light is collected in the direction of the exit hole. In the fluorescent light, a component that proceeds backward toward the entrance hole is reflected by the wavelength filter 268 and folded back in the direction of the exit hole. This folded back reflected component is combined with a component directly proceeding from the diffuser 272 toward the exit hole, forming final illumination light.

By such reflection functions of the holder 264 and wavelength filter 268, the intensity distributions and light distributions of the excitation light, fluorescent light, and non-excitation light at the exit hole are adjusted. If the intensity distribution is uneven or the light distribution is narrow when the illumination light enters the light guide 126, there is a risk that intensity unevenness or light distribution variation may occur in the illumination light that is guided to the distal end of the scope 100 by the light guide 126 and radiated to the observation object. In particular, in addition to the use of light having a plurality of wavelengths for the entire excitation light and non-excitation light, laser light with a narrow light distribution and fluorescent light with a wide light distribution are combined, so color unevenness is likely to occur. The occurrence of color unevenness in the illumination light will interfere with accurate observation. Therefore, it is necessary to sufficiently adjust the intensity distribution and light distribution of the illumination light when it enters the light guide 126.

In a normal lens system, such light distribution adjustment is difficult. In addition to a reason that light distribution adjustment by a lens is a wavelength-dependent in the first place, in the present invention, it is necessary to narrow the light distribution of fluorescent light whose original light distribution is wide and to widen the light distribution of excitation light and non-excitation light whose original light distribution is narrow. It is impossible for a lens system to achieve such a requirement on the same axis. In the embodiment, the diffuser 272 and reflector 266A (holder 264 and reflection coating 266) are used, and excitation light and non-excitation light are strongly diffused by the diffuser 272 so that their light distribution approaches the wide light distribution of fluorescent light, and then the intensity distributions and light distributions of the fluorescent light, excitation light, and non-excitation light are shaped tailored to the light guide 126 by the reflector 266A.

An optical effective diameter at an entrance of the light guide 126 is larger than an optical effective diameter at an exit of the wavelength conversion unit 260 (that is, the radius of the exit hole of the holder 264). In the light guide 126, it is desirable that a light acceptance angle represented by a numerical aperture NA is larger than spread angles of excitation light, non-excitation light, and fluorescent light. Satisfying such a magnitude relationship allows loss of the illumination light entering the light guide 126 from the wavelength conversion unit 260 to be reduced to the minimum.

<Connector Receiver 290>

The scope connector 190A and the connector receiver 290 are positioned so that the optical axis of the light guide lens 124 and the light guide 126 of the scope 100 is coincident with the wavelength conversion unit 260 when the scope connector 190A is attached to the connector receiver 290. Consequently, the optical axis of the light guide 126 at the entrance end that the illumination light enters is substantially coincident with the axis of the optical path of the illumination light emitted from the wavelength conversion unit 260.

<Protection Structure against Laser Light>

The optical path from the light source unit 220 to the wavelength conversion unit 260 is a part where laser light propagates in a space, and is located in the light source box 200A, which is a protection enclosure against laser light, from a viewpoint of guaranteeing eye safety to a user and the like.

Although the emission light from the wavelength conversion unit 260 includes the laser light from the laser light sources LD1-LD7 such as excitation light and non-excitation light, the laser light after being radiated to the diffuser 272 has lost coherence and is not dangerous.

<Illumination Device 400>

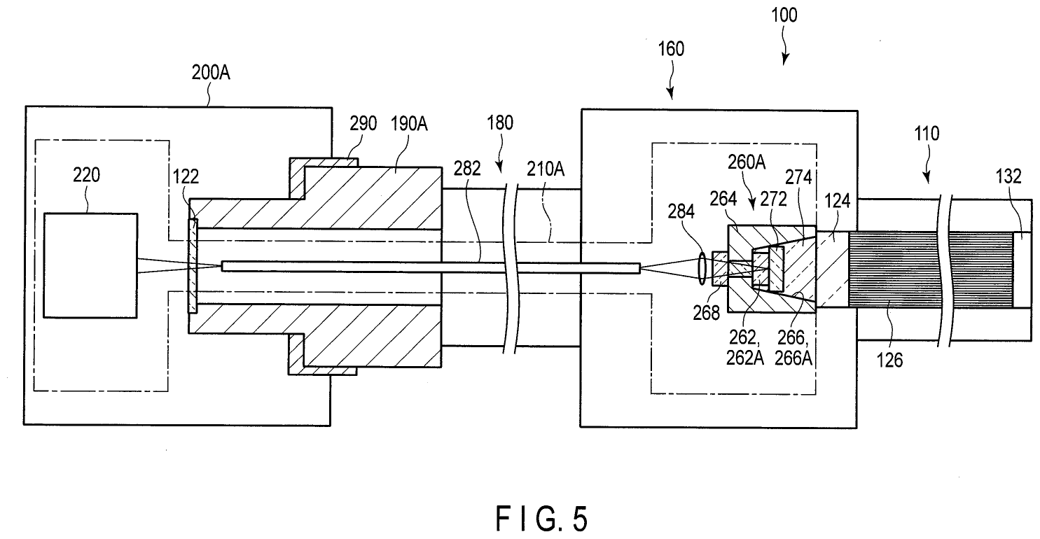

FIG. 5 schematically shows another configuration example different from the configuration example shown in FIG. 3 related to the illumination device 400 of the endoscope system shown in FIG. 2. In FIG. 5, members with the same reference signs as members shown in FIG. 3 are the same or similar members and their detailed description will be omitted.

The illumination device 400 according to this configuration example includes a light source device 210A configured to emit illumination light for illuminating the observation object, the light guide lens 124 that the illumination light emitted from the light source device 210A enters, the light guide 126 configured to guide the illumination light emitted from the light source device 210A, and the illumination light emission unit 132 configured to emit the illumination light guided by the light guide 126 to the outside of the scope 100.

The illumination light emission unit 132 is disposed at the distal end section 114 of the insertion section 110 of the scope 100 as in the configuration example of FIG. 3. However, the light guide lens 124 is disposed inside the control section 160 of the scope 100 different from the configuration example of FIG. 3. The light guide 126 extends inside the insertion section 110 from the light guide lens 124 to the illumination light emission unit 132.

The light source device 210A includes the light source unit 220 configured to be able to emit excitation light and non-excitation light, a light guide 282 configured to guide light emitted from the light source unit 220, a condenser lens 284 configured to converge laser light emitted from the light guide 282, and a wavelength conversion unit (wavelength converter) 260A configured to generate wavelength-converted light from excitation light that is emitted from the light guide 282 and enters through the condenser lens 284.

The light source unit 220 is disposed inside the light source box 200A as in the configuration example of FIG. 3. However, the wavelength conversion unit 260A is disposed inside the scope 100, for example, inside the control section 160 different from the configuration example of FIG. 3. The light guide 282 comprises a single optical fiber. The light guide 282 extends inside the connection cable 180 from the scope connector 190A to the inside of the control section 160.

An end of the light guide 282 situated on the side of the light source unit 220 is held by the scope connector 190A so as to be held at a constant position. An end of the light guide 282 situated on the side of the condenser lens 284 and the condenser lens 284 are held inside the control section 160 so as to be held at constant positions.

As compared with the illumination device 400 according to the configuration example of FIG. 3, the illumination device 400 according to the configuration example of FIG. 5 is structurally different with respect to the arrangement position of the wavelength conversion unit 260A, but is substantially the same with respect to optical functions and the like. That is, the illumination device 400 according to the configuration example of FIG. 5 optically operates substantially in the same way as the illumination device 400 according to the configuration example of FIG. 3.

<Illumination Device 400>

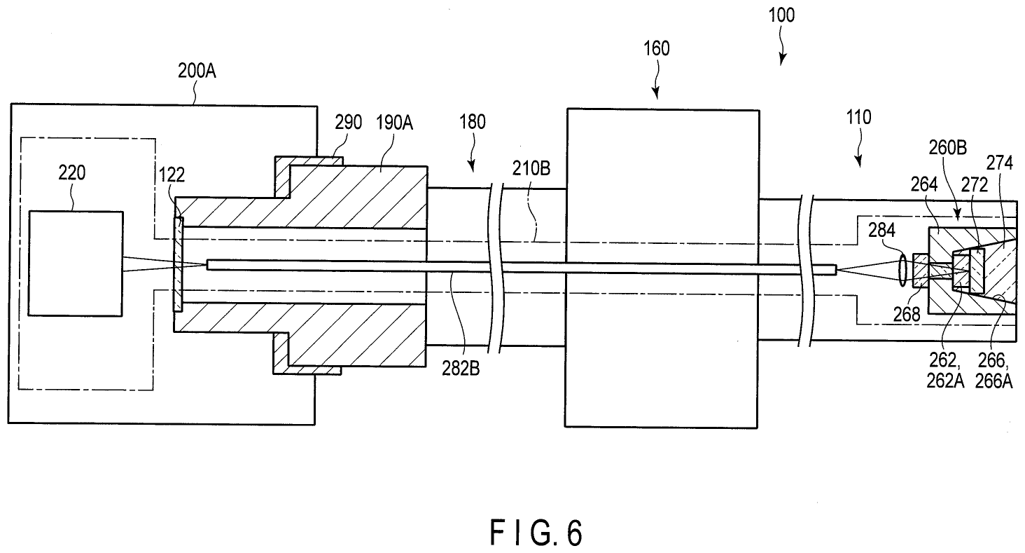

FIG. 6 schematically shows yet another configuration example different from the configuration example shown in FIG. 3 related to the illumination device 400 of the endoscope system shown in FIG. 2. In FIG. 6, members with the same reference signs as members shown in FIG. 3 or 5 are the same or similar members and their detailed description will be omitted.

In the illumination device 400 according to this configuration example, a light source device 210B configured to emit illumination light for illuminating the observation object includes the light source unit 220 configured to be able to emit excitation light and non-excitation light, a light guide 282B configured to guide the light emitted from the light source unit 220, the condenser lens 284 configured to converge laser light emitted from the light guide 282B, and a wavelength conversion unit (wavelength converter) 260B configured to generate wavelength-converted light from excitation light that is emitted from the light guide 282 and enters through the condenser lens 284.

The light source unit 220 is disposed inside the light source box 200A as in the configuration example of FIG. 3. However, the wavelength conversion unit 260B is disposed at the distal end section 114 of the insertion section 110 of the scope 100 different from the configuration example of FIG. 3. The light guide 282B comprises a single optical fiber. The light guide 282B extends from the scope connector 190A to a vicinity of the wavelength conversion unit 260B disposed at the distal end section 114 through the inside of the connection cable 180, control section 160, and insertion section 110.

An end of the light guide 282B situated on the side of the light source unit 220 is held by the scope connector 190A so as to be held at a constant position. An end of the light guide 282B situated on the side of the condenser lens 284 and the condenser lens 284B are held at the distal end section 114 of the insertion section 110 so as to be held at constant positions.

Different from the illumination devices 400 according to the configuration examples of FIGS. 3 and 5, the illumination device 400 according to the configuration example of FIG. 6 does not include the light guide 126 configured to guide illumination light emitted from the wavelength conversion unit 260 or 260A, and illumination light emitted from the wavelength conversion unit 260B is directly emitted to the outside of the scope 100 as is. In other words, the wavelength conversion unit 260B in this configuration example also has a function of the illumination light emission unit configured to emit illumination light to the outside of the scope 100.

[Illumination Mode]

In the light source device 210 of the above configurations, by combining excitation light and non-excitation light, in other words, switching the combination of the laser light sources LD1-LD7 to be turned on, a plurality of illumination modes suitable for observation purposes can be implemented. That is, the light source device 210 is configured to be able to drive in a plurality of illumination modes defining the spectrum of illumination light.

<White Light Illumination Mode>

In a white light illumination mode, the following two types of white light, namely fluorescent white light and laser white light, can be obtained by combining the laser light sources LD1-LD5, and those can be used in combination. That is, the white light illumination mode includes a mode of emitting fluorescent white light as illumination light, a mode of emitting laser white light as illumination light, and a mode of simultaneously emitting fluorescent white light and laser white light as illumination light.

(Fluorescent White Light: Blue Light+Fluorescence)

The laser light source LD2, a blue laser light source, is turned on. Components of part of excitation light, blue light emitted from the laser light source LD2, are absorbed by the fluorescent substance 262A, and fluorescent light is generated from the fluorescent substance 262A and emitted from the wavelength conversion unit 260. Part of blue light is transmitted without being absorbed by the fluorescent substance 262A, and is emitted from the wavelength conversion unit 260. By combining fluorescent light and blue light, fluorescent white light is obtained.

In addition to turning on the laser light source LD2, the laser light source LD1, a violet laser light source, and the laser light source LD5, a red laser light source, configured to each emit non-excitation light having a spectrum not overlapping with that of fluorescent light may be turned on together. By turning on the laser light sources LD1 and LD5 together in addition to the laser light source LD2, fluorescent white light much closer to natural white light is obtained.

In addition to turning on the laser light source LD2, the laser light source LD3, a green laser light source, and the laser light source LD4, an orange laser light source, may be turned on together. Since the spectra of green light and orange light entirely overlap with the spectrum of fluorescent light, by turning on the laser light sources LD3 and LD4 together in addition to the laser light source LD2, white light with increased components of green light and orange light compared with fluorescent white light is obtained.

Since fluorescent white light has a broad spectrum, it has a property of high color rendering. However, since the fluorescent substance 262A generates heat, it is difficult to obtain a large light quantity of fluorescent white light.

(Laser White Light: Violet Light+Green Light+Orange Light+Red Light)

The laser light source LD1, a violet laser light source, the laser light source LD3, a green laser light source, the laser light source LD4, an orange laser light source, and the laser light source LD5, a red laser light source, are simultaneously turned on. Thereby, violet light, green light, orange light, and red light are combined, and laser white light (or non-excitation white light) is obtained.

Since the spectrum of laser white light is discrete, laser white light has a property of low color rendering. However, since the fluorescent substance 262A does not generate heat, it is easy to obtain a large light quantity of laser white light.

Note that combinations of non-excitation light for obtaining laser white light (or non-excitation white light) are not limited to the combination of violet light, green light, orange light, and red light. As long as light that can be substantially regarded as white light can be obtained, any combination of non-excitation light may be applied to generation of non-excitation white light. For example, as long as this requirement is met, a combination of only two rays of non-excitation light may be applied to generation of non-excitation white light.

(Combination of Fluorescent White Light and Laser White Light)

For example, the light source device 210 is driven as follows. The laser light sources LD1-LD5 are simultaneously turned on. Thereby, illumination light including fluorescent white light and laser white light is obtained. In other words, the light source device 210 is driven in the white light illumination mode in which fluorescent white light obtained by combining excitation light and fluorescent light and non-excitation white light obtained by combining a plurality of rays of non-excitation light are simultaneously emitted as illumination light. The ratio of quantities of light of fluorescent white light and laser white light can be changed, for example, by adjusting drive currents of the laser light source LD2 and the other laser light sources LD1, LD3, LD4, and LD5. For example, for observation requiring the color rendering property, the light source device 210 is driven in a high color rendering white light illumination mode in which illumination light with a high ratio of fluorescent white light is emitted, and for observation requiring a large quantity of light, the light source device 210 is driven in a large light quantity white light illumination mode in which illumination light with a high ratio of laser white light is emitted.

In other words, the white light illumination mode includes the high color rendering white light illumination mode in which in the quantity of light emitted from the wavelength conversion unit 260, a total light quantity of excitation light and fluorescent light is larger than a total light quantity of a plurality of rays of non-excitation light, and the large light quantity white light illumination mode in which the total light quantity of the excitation light and the fluorescent light is smaller than the total light quantity of the plurality of rays of non-excitation light.

Laser white light can achieve a large light quantity but the color rendering property is lowered. Although there is a method of correcting an impact on an acquired image due to reduction in the color rendering property by image processing, it is difficult to estimate and correct information on wavelength components that are not included at all in the spectrum of illumination light. Therefore, by mixing fluorescent white light into laser white light even if its ratio is low, information on a wavelength range that is lost in laser white light is supplemented by fluorescent light. In this way, including fluorescent white light and laser white light in illumination light and combining with correction image processing allows an image with high image quality and high color rendering to be obtained.

That is, in observation by the large light quantity white light illumination mode, it is good to apply correction using image information obtained based on fluorescent light of illumination light to image information obtained based on excitation light and non-excitation light of the illumination light.

<Special Light Illumination Mode>

In a special light illumination mode, the following special light can be obtained by combinations of the laser light sources LD1-LD7.

(Violet Light+Green Light)

The laser light source LD1, a violet laser light source, and the laser light source LD3, a green laser light source, are simultaneously turned on. Thereby, special light including violet light (wavelength 390-445 nm) and green light (wavelength 530-550 nm) is obtained. This special light is suitable for narrow band imaging (NBI). In narrow band imaging using this special light, blood vessels from a surface layer to a middle layer that is between the surface layer and a deep layer are emphasized and observed.

(Violet Light+Green Light)

The laser light source LD1, a violet laser light source, and the laser light source LD3, a green laser light source, are simultaneously turned on. Thereby, special light including violet light (wavelength 390-470 nm) and green light (wavelength 540-560 nm) is obtained. This special light is suitable for auto fluorescent light imaging (AFI).

(First Infrared Light+Second Infrared Light)

The laser light source LD6, a first infrared laser light source, and the laser light source LD7, a second infrared laser light source, are simultaneously turned on. Thereby, special light including first infrared light (wavelength 790-820 nm) and second infrared light (wavelength 905-970 nm) is obtained. This special light is suitable for infrared imaging (IRI) that acquires information on internal blood vessels.

(Blue Light+Orange Light+Red Light)

The laser light source LD2, a blue laser light source, the laser light source LD4, an orange laser light source, and the laser light source LD5, a red laser light source, are simultaneously turned on. Thereby, special light including fluorescent white light, orange light (wavelength 600-630 nm), and red light (wavelength 680-700 nm) is obtained.

(White Light+Infrared Light)

The laser light source LD2, a blue laser light source, the laser light source LD6, a first infrared laser light source, and the laser light source LD7, a second infrared laser light source, are simultaneously turned on. Thereby, special light including fluorescent white light, first infrared light, and second infrared light is obtained.

Alternatively, the laser light source LD1, a violet laser light source, the laser light source LD3, a green laser light source, the laser light source LD4, an orange laser light source, the laser light source LD5, a red laser light source, the laser light source LD6, a first infrared laser light source, and the laser light source LD7, a second infrared laser light source, are simultaneously turned on. Thereby, special light including laser white light, first infrared light, and second infrared light is obtained.

This special light is suitable for angiography and blood flow observation by indocyanine green (ICG).

(Violet Light+Infrared Light)

The laser light source LD1, a violet laser light source, and the laser light source LD7, an infrared laser light source, are simultaneously turned on. Thereby, special light including violet light (wavelength 390-445 nm) and infrared light (wavelength 905-970 nm) is obtained. Instead of the laser light source LD7, the laser light source LD6, which is also an infrared laser light source, may be turned on.

Furthermore, the laser light source LD2, a blue laser light source, may simultaneously be turned on. Thereby, special light including fluorescent white light, violet light, and infrared light is obtained. Since the wavelength bands of violet light and infrared light are out of the wavelength band of fluorescent light, it is possible to perform special light observation such as blood vessel enhancement by non-excitation light while maintaining the hue of fluorescent white light.

(Violet Light+Green Light+Red Light)

The laser light source LD1, a violet laser light source, the laser light source LD3, a green laser light source, and the laser light source LDS, a red laser light source, are simultaneously turned on. Thereby, special light including violet light (wavelength 390-445 nm), green light (wavelength 530-550 nm), and red light (wavelength 680-700 nm) is obtained. This special light is suitable for narrow band imaging. In narrow band imaging using this special light, blood vessels from the surface layer to the deep layer situated deeper than the middle layer are emphasized and observed.

(Other Combination of Non-excitation Light)

It is also possible to obtain special light different from the special light described above by simultaneously turning on non-excitation light sources in combinations other than the combinations of non-excitation light sources described above. Combinations of non-excitation light sources simultaneously turned on may be determined according to observation purposes, objects, and the like.

Although the above is described on the presumption that the excitation light source and non-excitation light sources used for illumination are simultaneously turned on, a display image may be formed by sequentially turning on those light sources and combining a plurality of images taken in synchronization with each lighting timing.

As will be understood from the above, the special light illumination mode includes the high color rendering special light illumination mode in which light obtained by combining at least one ray of non-excitation light with fluorescent white light obtained by combining excitation light and fluorescent light is emitted as illumination light and the large light quantity special light illumination mode in which light including only at least one ray of non-excitation light is emitted as illumination light.

[Effect]

As described above, in the light source device 210 according to the embodiment, the optical paths of laser light emitted from the laser light sources LD1-LD7 are integrated into one common optical path by the optical combiner 230. As a result, it is also applicable to a case where the scope 100 is a scope including a thin insertion section.

In addition, it is possible to emit various rays of illumination light having different wavelength components by changing combinations of the laser light sources LD1-LD7 to be turned on. Therefore, it is possible to cope with various observation methods. Furthermore, it is considered that it can cope with not only known observation methods but also unknown observation methods.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
at least one excitation light source configured to emit excitation light;
at least two non-excitation light sources configured to respectively emit at least two rays of non-excitation light;
an optical combiner configured to integrate an optical path of the excitation light and optical paths of the non-excitation light into a common optical path; and
a wavelength conversion unit including a wavelength conversion member disposed on the common optical path,
the wavelength conversion member being configured to: transmit the non-excitation light; absorb components of part of the excitation light to generate wavelength-converted light having a wavelength different from a wavelength of the excitation light; and emit illumination light including the transmitted non-excitation light and the generated wavelength-converted light,
spectra of the at least two rays of non-excitation light each having a peak wavelength in a wavelength band out of a wavelength band of a spectrum of the wavelength-converted light.

2. The endoscope system according to claim 1, wherein
a peak wavelength of a spectrum of at least one ray of non-excitation light of the at least two rays of non-excitation light is shorter than a peak wavelength of the spectrum of the wavelength-converted light, and a peak wavelength of a spectrum of at least one other ray of non-excitation light of the at least two rays of non-excitation light has the peak wavelength of the spectrum of the wavelength-converted light.

3. The endoscope system according to claim 1, wherein
a peak wavelength of a spectrum of at least one ray of non-excitation light of the at least two rays of non-excitation light is shorter than a peak wavelength of a spectrum of the excitation light and shorter than a longest wavelength of the spectrum of the wavelength-converted light, and a peak wavelength of at least one other ray of non-excitation light of the two rays of non-excitation light is longer than the peak wavelength of the spectrum of the excitation light and shorter than the longest wavelength of the spectrum of the wavelength-converted light.

4. The endoscope system according to claim 1, wherein
the at least two non-excitation light sources comprises at least three non-excitation light sources configured to emit at least three rays of non-excitation light having different wavelengths, and
the wavelength conversion member is configured to emit the illumination light including the at least three rays of non-excitation light.

5. The endoscope system according to claim 1, wherein
the optical combiner is configured to integrate the optical path of the excitation light and the optical paths of the non-excitation light so that their optical axes are substantially coincident and their diameters are substantially coincident.

6. The endoscope system according to claim 1, wherein
the wavelength conversion member comprises a fluorescent substance and is configured to absorb components of part of the irradiated excitation light to generate fluorescent light having a wavelength longer than the wavelength of the excitation light, and transmittance of the wavelength conversion member is higher in wavelength regions of the non-excitation light than in a wavelength region of the excitation light.

7. The endoscope system according to claim 6, wherein
the wavelength conversion unit includes a wavelength filter configured to transmit the excitation light and the non-excitation light and reflect the fluorescent light at a portion that the excitation light and the non-excitation light enter.

8. The endoscope system according to claim 6, wherein
the endoscope system is configured to be driven in illumination modes that define a spectrum of the illumination light, and the illumination modes include a white light illumination mode in which at least one of fluorescent white light composed of the excitation light and the fluorescent light, and non-excited white light composed of the at least two rays of non-excitation light is emitted as the illumination light.

9. The endoscope system according to claim 6, wherein
the endoscope system is configured to be driven in illumination modes that define a spectrum of the illumination light, and the illumination modes include a white light illumination mode in which fluorescent white light composed of the excitation light and the fluorescent light, and non-excited white light composed of the at least two rays of non-excitation light are simultaneously emitted as the illumination light.

10. The endoscope system according to claim 9, wherein
the white light illumination mode can be switched to one of:
a high color rendering white light illumination mode in which a total light quantity of the excitation light and the fluorescent light is larger than a total light quantity of the at least two rays of non-excitation light in a quantity of light emitted from the wavelength conversion unit; and
a large light quantity white light illumination mode in which the total light quantity of the excitation light and the fluorescent light is smaller than the total light quantity of the at least two rays of non-excitation light in the quantity of light emitted from the wavelength conversion unit.

11. The endoscope system according to claim 1, wherein
the wavelength conversion member comprises a fluorescent substance configured to absorb components of part of the excitation light to generate a fluorescent light,
the endoscope system is configured to be driven in illumination modes that define a spectrum of the illumination light, the illumination modes include at least one of:
a high-color-rendering special light illumination mode in which light composed of at least one ray of non-excitation light with fluorescent white light composed of the excitation light and the fluorescent light is emitted as the illumination light; and
a large light quantity special light illumination mode in which light consisting only of at least one ray of non-excitation light is emitted as the illumination light.

12. The endoscope system according to claim 1, comprising
a scope configured to at least illuminate an observation target, the scope including a light guide configured to guide the illumination light emitted from the wavelength conversion unit, the wavelength conversion unit is disposed outside the scope.

13. The endoscope system according to claim 1, comprising
a scope configured to at least illuminate an observation target, the scope emitting the illumination light emitted from the wavelength conversion unit toward the observation target, the wavelength conversion unit is disposed inside the scope.

14. The endoscope system according to claim 12, wherein
an optical axis of the light guide at an entrance end that the illumination light enters is substantially coincident with an axis of an optical path of the illumination light emitted from the wavelength conversion unit,
an optical effective diameter at an entrance of the light guide is larger than an optical effective diameter at an exit of the wavelength conversion unit,
in the light guide, an acceptance angle of light represented by the numerical aperture NA is larger than spread angles of the excitation light, the non-excitation light, and the wavelength-converted light, and
the light guide comprises a bundle fiber in which a number of optical fibers are bundled.

15. A light source device for endoscopes comprising:
at least one excitation light source configured to emit excitation light;
at least one non-excitation light source configured to emit non-excitation light;
an optical combiner configured to integrate an optical path of the excitation light and an optical path of the non-excitation light into a common optical path; and
a wavelength conversion unit including a wavelength conversion member disposed on the common optical path, and a reflector configured to control light distributions of the excitation light, the non-excitation light, and the wavelength-converted light,
the wavelength conversion member being configured to: transmit the non-excitation light, absorb components of part of the excitation light to generate wavelength-converted light having a wavelength different from a wavelength of the excitation light; and emit illumination light including the transmitted non-excitation light and the generated wavelength-converted light,
the reflector being configured to cause the light distributions of the excitation light, non-excitation light, and wavelength-converted light emitted from the wavelength conversion unit to be equal to or less than a predetermined spread angle.

16. The light source device for endoscopes according to claim 15, further comprising
a diffuser configured to diffuse the incoming excitation light and the incoming non-excitation light to expand the light distributions of the excitation light and the non-excitation light, the diffused light comprising a forward-scattered light component and a backscattered light component,
the reflector being configured to reflect the backscattered light component, combining it with the forward-scattered light component, so as to cause a light distribution of the diffused light emitted from the wavelength conversion unit to be equal to or less than a predetermined spread angle.

17. The light source device for endoscopes according to claim 16, wherein
the wavelength conversion member comprises a fluorescent substance configured to absorb components of part of the irradiated excitation light to generate fluorescent light having a wavelength longer than the wavelength of the excitation light, the wavelength-converted light comprising the fluorescent light, the fluorescent light comprising a forward fluorescent light component and a rearward fluorescent light component,
the reflector is configured to reflect the rearward fluorescent light component, combining it with the front fluorescent light component, so as to cause the light distribution of the fluorescent light emitted from the wavelength conversion unit to be equal to or less than a predetermined spread angle, and
the reflector substantially matches the light distributions of the excitation light, non-excitation light, and fluorescent light emitted from the wavelength conversion unit.

18. The light source device for endoscopes according to claim 15, wherein
the wavelength conversion unit is optically connected to a scope including a light guide configured to guide incoming light, and
an optical effective diameter on an exit side of the wavelength conversion unit is equal to or smaller than an optical effective diameter on an entrance side of the light guide.
